(19) United States
(12) Patent Application Publication
(10) Pub. No.: US 2007/0287912 A1
(43) Pub. Date: Dec. 13, 2007

(54) Functional imaging using capacitive micromachined ultrasonic transducers

(76) Inventors: Butrus T. Khuri-Yakub, Palo Alto, CA (US); Omer Oralkan, Santa Clara, CA (US); Ira O. Wygant, Palo Alto, CA (US); Srikant Valthllingam, Palo Alto, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/803,157

(22) Filed: May 11, 2007

Related U.S. Application Data
(60) Provisional application No. 60/810,106, filed on May 31, 2006.

(51) Int. Cl. A61B 8/00; A61N 5/067

(52) U.S. Cl. 600/439; 367/181; 600/437; 600/443; 600/459; 600/471; 607/93

(57) ABSTRACT

The present invention provides an apparatus for functional imaging of an object that is compact, sensitive, and provides real-time three-dimensional images. The apparatus includes a source of non-ultrasonic energy, where the source induces generation of ultrasonic waves within the object. The source can provide any type of non-ultrasonic energy, including but not limited to light, heat, microwaves, and other electromagnetic fields. Preferably, the source is a laser. The apparatus also includes a single capacitive micromachined ultrasonic transducer (CMUT) device or an array of CMUTs. In the case of a single CMUT element, it can be mechanically scanned to simulate an array of any geometry. Among the advantages of CMUTs are tremendous fabrication flexibility and a typically wider bandwidth. Transducer arrays with high operating frequencies and with nearly arbitrary geometries can be fabricated. A method of functional imaging using the apparatus is also provided.

### CROSS-REFERENCE TO RELATED APPLICATIONS

This application claims priority from U.S. Provisional Patent Application No. 60/810,106, filed May 31, 2006, which is incorporated herein by reference.

### STATEMENT REGARDING FEDERALLY SPONSORED RESEARCH OR DEVELOPMENT

This invention was supported in part by grant number 5R33CA099059-03 from the National Institutes of Health (NIH). The U.S. Government has certain rights in the invention.

### FIELD OF THE INVENTION

The present invention relates generally to medical imaging. More particularly, the present invention relates to functional imaging using capacitive micromachined ultrasonic transducers.

### BACKGROUND

Traditional ultrasound images are formed by first transmitting ultrasound to a medium of interest and then receiving the ultrasound signals resulting from the interaction of the transmitted signals with the medium. This kind of an image is usually a representation of the mechanical properties of the medium and provides structural or anatomical information. The interaction of the medium with other forms of energy can provide additional information about the functional differences even in a structurally indifferent, uniform medium. For instance, when a short laser pulse is transmitted into a tissue, the introduced light energy is absorbed and scattered in a different manner by different parts of the tissue. The optical absorption depends on the wavelength of the light and the properties of the medium at the molecular or even atomic level. Regions with stronger absorption characteristics in a tissue generate stronger acoustic signals via the thermoelastic effect, which is simply the thermal expansion of the imaging regions resulting in a mechanical disturbance and hence an acoustic signal. By collecting these light-induced acoustic signals using a transducer or array of transducers, one can construct an image that is a representation of the light absorption characteristics of the sample. One example of this approach is to image the microvasculature in tissue by detecting blood oxygenation, which is usually a sign of angiogenesis indicating a cancerous lesion. In this example, the increased light absorption of the oxygenated blood is used to create a high-contrast image.

Existing functional ultrasound imaging methods are based on mechanically scanned single transducers, or the combination of a laser source with a one-dimensional commercial imaging probe. These approaches do not provide real-time three-dimensional images. In addition, current devices are bulky and not suitable for intracavital applications.

Furthermore, existing systems are based on piezoelectric transducer technology. Using piezoelectric transducer technology, it is difficult to fabricate arrays of highly performing transducer elements when the array geometry is not rectilinear (for example, a ring array) and for high transducer operating frequencies. Accordingly, there is a need in the art to develop a method and apparatus for functional ultrasound imaging that is small, that provides three-dimensional images in real time, and that can accommodate many types of geometries.

### SUMMARY OF THE INVENTION

The present invention provides an apparatus for functional imaging of an object that is compact, sensitive, and provides real-time three-dimensional images. The apparatus includes a source of non-ultrasonic energy, where the source induces generation of ultrasonic waves within the object. The source can provide any type of non-ultrasonic energy, including but not limited to light, heat, microwaves, and other electromagnetic fields. Preferably, the source is a laser. The apparatus also includes a single capacitive micromachined ultrasonic transducer (CMUT) device or an array of CMUTs. In the case of a single CMUT element, it can be mechanically scanned to simulate an array of any geometry. Among the advantages of CMUTs are tremendous fabrication flexibility and a typically wider bandwidth. Transducer arrays with high operating frequencies and with nearly arbitrary geometries can be fabricated. The wider bandwidth of CMUTs provides better image resolution and potential for novel imaging methods.

CMUT arrays according to the present invention may have any configuration, such as a 1-dimensional array, a 2-dimensional array, or an annular or ring array. Preferably, the array has elements that measure along one dimension (both dimensions for two-dimensional arrays) about one-half the wavelength of the ultrasound being measured. The total size of the array is preferably large enough to provide sufficient signal-to-noise ratio and resolution for a given application. Also preferably, the array or single CMUT includes integrated circuitry.

The present invention also provides a method of functionally imaging an object. The method includes the steps of exposing the object to a source of non-ultrasonic energy, where the source induces generation of ultrasonic waves in the object, and detecting the generated ultrasonic waves with a CMUT device.

### BRIEF DESCRIPTION OF THE FIGURES

The present invention together with its objectives and advantages will be understood by reading the following summary in conjunction with the drawings, in which:

FIG. 1 shows examples of array configurations according to the present invention.

FIG. 2 shows examples of configurations of an apparatus according to the present invention.

FIG. 3 shows possible positions of the non-ultrasonic excitation relative to the imaging field according to the present invention.

FIG. 4 shows a schematic of functional imaging according to the present invention.

FIG. 5 shows a schematic of a setup for an experiment using an apparatus according to the present invention.

FIG. 6 shows data obtained using an apparatus according to the present invention.

FIG. 7 shows images obtained using an apparatus according to the present invention.

FIG. 8 shows results of an experiment demonstrating the sensitivity of an apparatus according to the present invention.

### DETAILED DESCRIPTION OF THE INVENTION

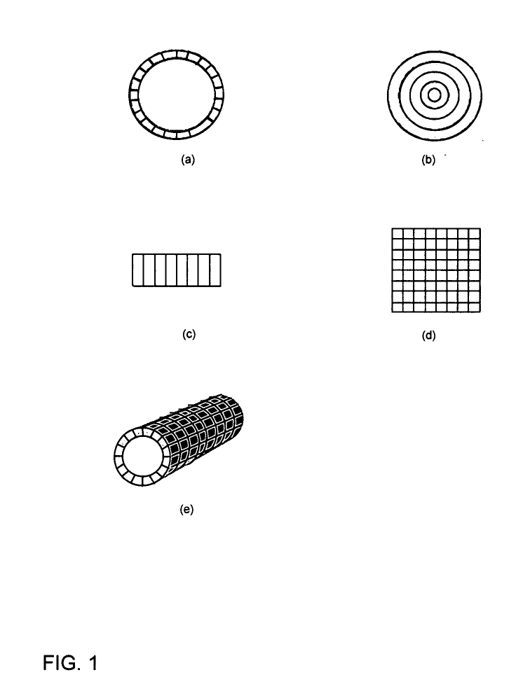

The present invention provides an apparatus for functional ultrasound imaging of an object, including a source of non-ultrasonic excitation energy and a single CMUT or an array of CMUTs. The source may be any type of source, including but not limited to light (with different wavelengths depending on the absorption characteristics of the imaging target), rapid thermal heating, microwaves, radio-frequency (RF) electromagnetic waves and other electromagnetic fields, electron beams, etc., but is preferably a laser. The CMUT arrays may be in any type of configuration. FIG. 1 shows examples of array configurations according to the present invention, including an annular ring array (FIG. 1(a)), an annular array (FIG. 1(b)), a one-dimensional linear array (FIG. 1(c)), a two-dimensional rectangular array (FIG. 1(d)) and a cylindrical array (FIG. 1(e)). CMUT arrays may also be formed on a curved surface. In addition, arrays may be formed around the target object to allow tomographic image reconstruction methods. A single CMUT or multiple CMUTs can be mechanically scanned to simulate an array with more elements.

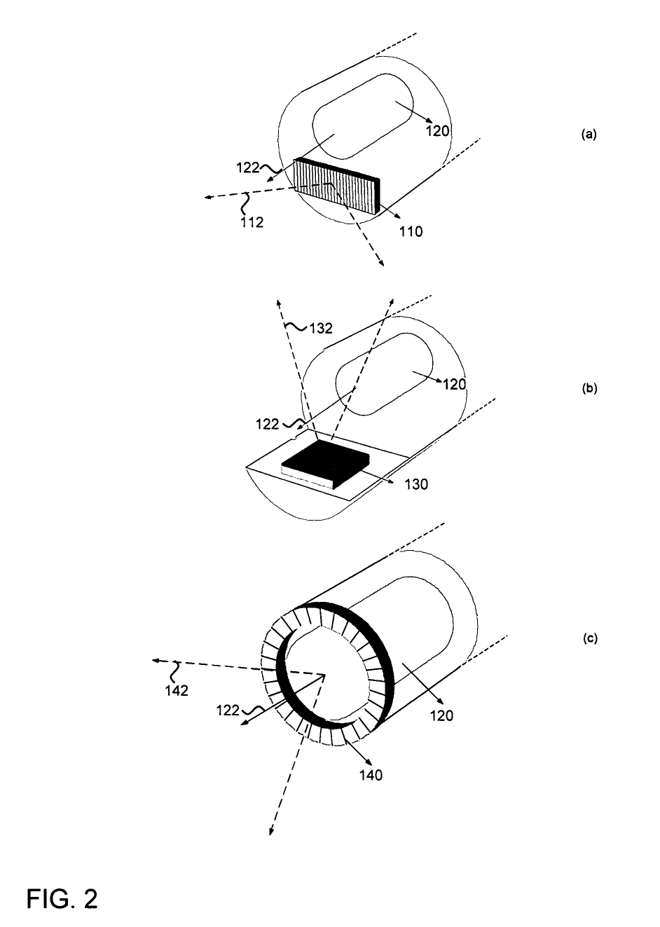

Several apparatus designs are possible according to the present invention, based on different types of non-ultrasonic radiation sources and CMUT arrays with different geometries. For medical applications, these apparatuses can be used externally or from within the body. Some sample designs for functional ultrasonic imaging apparatuses employing a laser excitation and a CMUT array are shown in FIG. 2. FIG. 2 (a) shows an apparatus with a linear CMUT array 110 in conjunction with an optical fiber 120 to provide a short laser pulse in the form of laser beam 122. This apparatus has an imaging field indicated by dashed lines 112. This type of apparatus provides a two-dimensional cross-sectional image. To obtain a volume image with this kind of apparatus requires mechanical scanning. A real-time three-dimensional functional image can be acquired by using a two-dimensional aperture that can be electronically scanned. One example of such an apparatus is shown in FIG. 2 (b). This apparatus again has an optical fiber 120 to provide a short laser pulse 122. This apparatus employs a two-dimensional rectangular array 130, which provides an imaging field, indicated by dashed lines 132, which is perpendicular to the laser beam 122. The array can also be used in parallel with the laser beam 122. Such an approach is shown in FIG. 2 (c) where an annular ring array 140, with imaging field indicated by dashed lines 142, is used to form a real-time three-dimensional functional image. The internal cavity of the array 140 is occupied by the optical fiber 120 to provide the laser pulse 122. Another advantage of the ring array is that the working channel can contain not only the optical fiber that brings in the light beam, but also may bring in a therapeutic device to burn an occlusion, scissors to extract a piece of tissue, or any other needed working tool. The arrays depicted in these sample designs can be integrated with supporting integrated circuits to improve the overall image quality. These examples are provided to help visualize the general approach according to the invention and are not meant to describe all possibilities.

In one embodiment of the invention, a silicon substrate is used to allow the described non-ultrasonic energy sources to be integrated on the same substrate with the CMUT array. Vertical cavity surface emitting lasers, microfabricated electron beam sources, and nanokylstrons for microwave generation are examples of sources that may be integrated with the CMUT array.

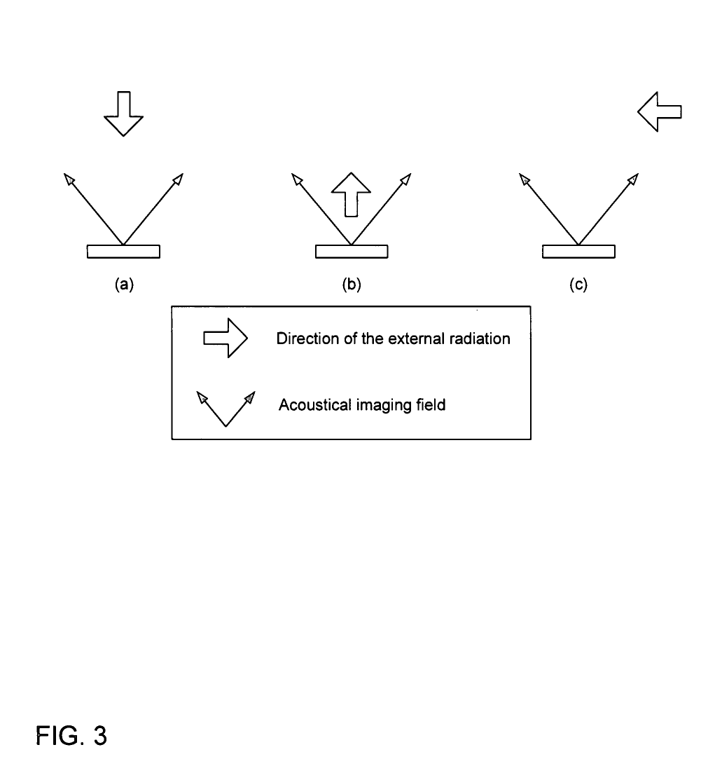

The excitation energy can be applied from different directions and by different means. FIG. 3 shows that the non-ultrasonic excitation can be applied from the opposite side of the CMUT array, or in the same direction or perpendicular to the array. For external applications the excitation energy can be provided in free space, whereas for intracavital applications, such as intravascular, transvaginal and transrectal applications, using a waveguide is more appropriate. Internal use of these apparatuses also includes other catheter based, endoscopic or laparoscopic applications.

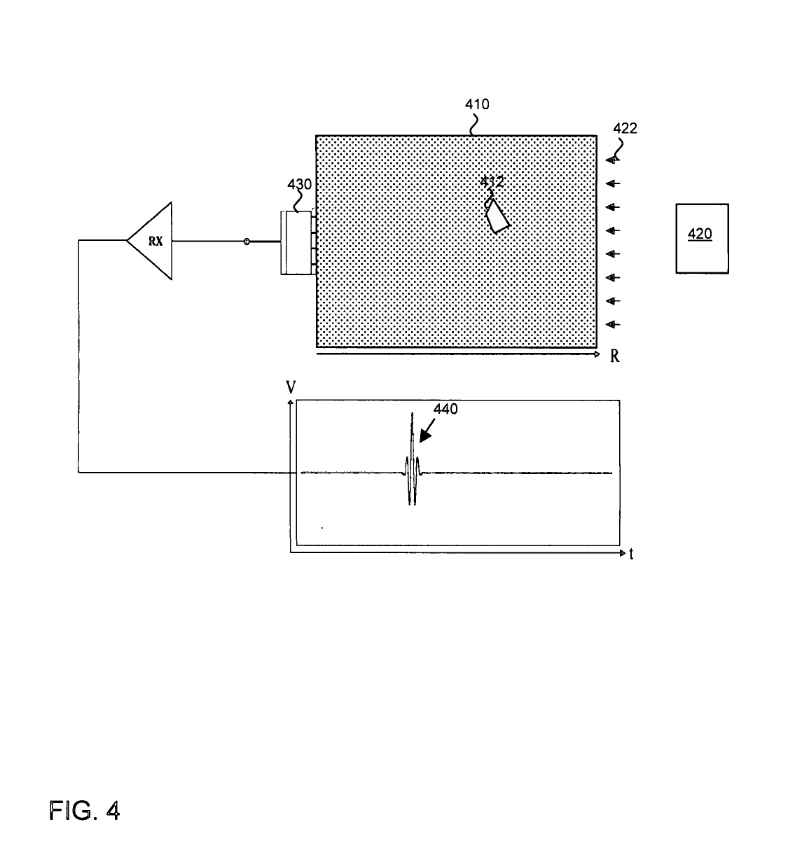

The present invention also provides a method of functionally imaging an object, including the steps of exposing the object to a source of non-ultrasonic energy, generating ultrasonic waves in the object, and detecting the ultrasonic waves in the object. This method is shown schematically in FIG. 4. Object 410, with high absorption region 412, is exposed to non-ultrasonic excitation energy, indicated by arrows 422, from source 420. The non-ultrasonic energy then generates ultrasound waves in the object 410. These waves are in turn detected by CMUT array 430. The received signal 440 is an indication of a strong absorber of the non-ultrasonic excitation energy.

According to the present invention, the functional imaging method may be used alone or in addition to conventional ultrasound imaging to map the functionality to the anatomy. When used in conjunction with conventional ultrasound imaging, the ultrasound waves may be transmitted through the object and detected using one or more of the CMUTs of the array. In one embodiment, the inventive functional imaging method is time multiplexed with conventional ultrasound, thus allowing the two signals to be differentiated. The ultrasound signals may then be processed to form images from the detected generated ultrasound waves and the detected transmitted ultrasound waves. These images may be displayed either separately or as overlapping images, using techniques known in the art.

In one embodiment, the induced acoustic signal intensity can be observed as a function of the excitation frequency. Different ultrasound images can then be reconstructed at each frequency of excitation, to implement a functional equivalent of a spectroscope.

The excitation energy can also be used for therapeutic applications. For example, the design described in FIG. 2(c) could be used for both photoacoustic imaging and tissue ablation by increasing the power level of the laser source. Similarly, microwaves and RF fields can be used for ablation of tissue. The method of the present invention may also be used to monitor the therapy, such as the extent and the nature of the lesion resulting from the ablation procedure. Other uses of the present invention are applications such as non-destructive testing and acoustic microscopy.

In one embodiment of the present method, a coded excitation scheme is used, using methods known in the art. In this embodiment, e.g., a laser pulse or RF excitation is coded. When the received ultrasound signal is decoded during image reconstruction, an improvement in the overall signal and image quality can be obtained.

Contrast enhancing biocompatible dyes, micro- or nano-particles (metal or organic material based), or other molecular probes can be used along with the proposed method. Coating or conjugating micro- or nano-particles with custom designed materials or molecules will provide attachment to certain targeted cells or tissues. Similarly, different molecules can be engineered to act as a contrast agent by attaching to specific target tissues, e.g., a tumor. If these particles or molecules are designed to absorb the external energy at certain wavelengths, the image contrast can be enhanced. By changing the particle size and material properties, the wavelength of the induced ultrasound can also be adjusted.

### EXAMPLES

The present invention has been demonstrated with photoacoustic imaging. Details on this demonstration may be found in “Capacitive Micromachined Ultrasonic Transducers (CMUTs) for Photoacoustic Imaging”, by Vaithilingam et al., Proceedings of SPIE vol. 6086, 608603, 1-11, 2006; and “Photoacoustic Imaging Using a Two-Dimensional CMUT Array”, by Wygant et al., Proc. of 2005 IEEE Ultrasonics Symposium, 1921-1924, both of which are incorporated by reference herein. A brief description of these experiments follows:

Experimental Setup

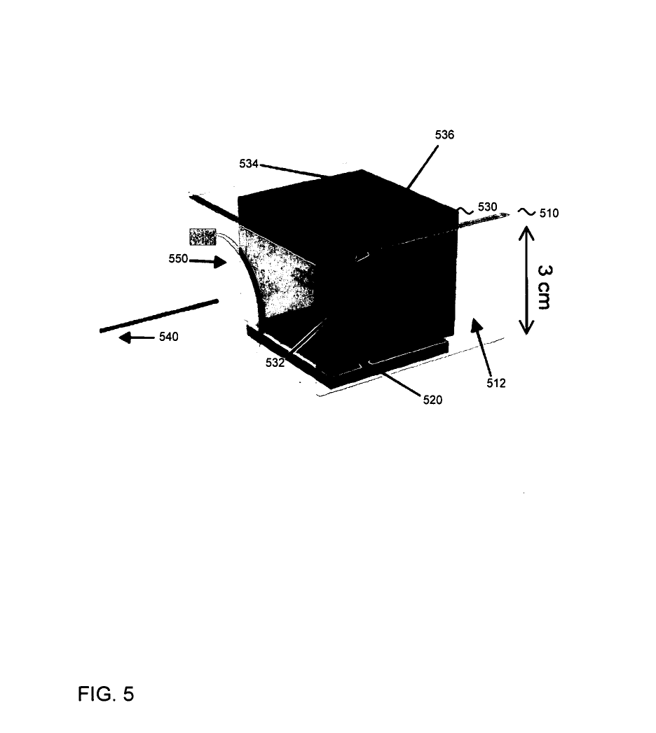

A diagram illustrating the experimental setup is shown in FIG. 5. For these experiments, the phantom to be imaged is suspended in an oil tank 510 of size 5 cm×5 cm×3 cm. Vegetable oil 512 is used to couple ultrasound between the array and electronics 520 and phantom 530. Vegetable oil is used because it is nonconducting and thus the array and electronics 520 do not need to be insulated. By insulating the electronics and array, conductive mediums such as water can be imaged. The phantom 530 is made of three 0.86-mm inner diameter (1.27-mm outer diameter) polyethylene tubes 532 passing through a 2 cm×2 cm×3 cm block of tissue mimicking material 534 (ATS Laboratories, Bridgeport, Conn.). The center tube 536 is filled with India-ink to provide optical contrast for the photoacoustic imaging. The CMUT array and electronics 520 are located at the bottom of the tank 510. The phantom is illuminated from the side of the tank by a Q-switched Nd:YAG laser 540. Ideally the laser 540 should uniformly illuminate the material being imaged. Thus the laser beam is de-focused to a 1/e² diameter of approximately 6 mm. A ground glass diffuser 550 in front of the tank 510 further diffuses the laser light. The laser used has a 1.064 μm wavelength and 12-ns FWHM pulse duration. The energy of each laser pulse is 2.3 mJ. The laser was fired at a rate of 10 Hz.

CMUT Array Tiling

CMUT technology allows the fabrication of large two-dimensional arrays. The advantages of larger arrays include the ability to image larger targets with an improved signal to noise ratio. Larger arrays also result in improved lateral resolution due to a larger aperture size. To simulate this effect, array tiling was performed. In our experiment the CMUT array was placed on an X-Y translational stage. After one data set was acquired, the array was translated 4 mm (length of the array) along the x-direction and another data set was acquired. Further data sets were obtained by also translating 4 mm along the y-direction. In all, 9 data sets were acquired. Hence, the intention is that array tiling will result in an image that will be equivalent to an image taken with an array of size 48×48 elements.

CMUT Array and Integrated Electronics

The transducer array has 256 elements (16×16 elements). Each element is 250 μm×250 μm. Thus, the entire array size is 4 mm×4 mm. The transducers have a center frequency of 5 MHz. The CMUT array was fabricated using surface micromachining with membranes made of silicon nitride. A few of the key CMUT device parameters are shown in Table 1. A more thorough description of the design and fabrication of the CMUT array has been reported elsewhere. A description of the CMUT array and integrated electronics has also been previously reported. The transducer array is flip-chip bonded to a custom-designed integrated circuit (IC) that comprises the front-end circuitry. The result is that each element is connected to its own amplifier via a 400-μm long through-wafer via. Integrating the electronics in this manner mitigates the effect of parasitic cable capacitance and simplifies connecting the transducer array to an external system. The IC allows for the selection of a single element at a time. Thus, 256 pulses are required to acquire a single image with no averaging. For a propagation limited system, this allows a maximum achievable frame rate of 100 frames/sec for imaging a 3-cm volume in oil.

TABLE 1
CMUT Device Parameters

| | |
|---|---|
| Cell diameter, μm | 36 |
| Element pitch, μm | 250 |
| Number of cells per element | 24 |
| Membrane thickness, μm | 0.6 |
| Cavity thickness, μm | 0.1 |
| Insulating layer thickness, μm | 0.15 |
| Silicon substrate thickness, μm | 400 |
| Flip-chip bond pad diameter, μm | 50 |
| Through-wafer interconnect diameter, μm | 20 |

Results

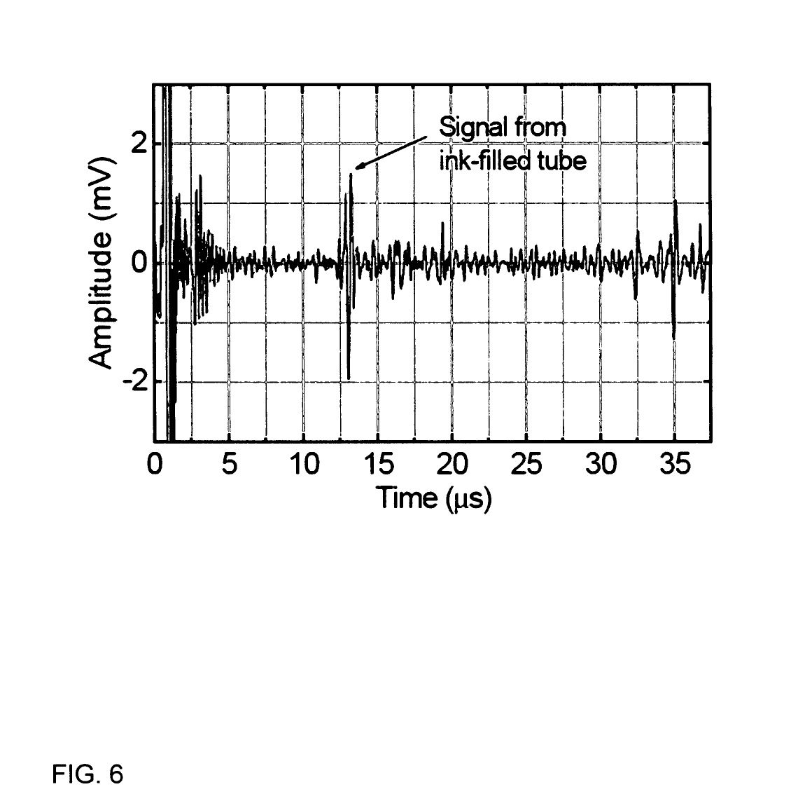

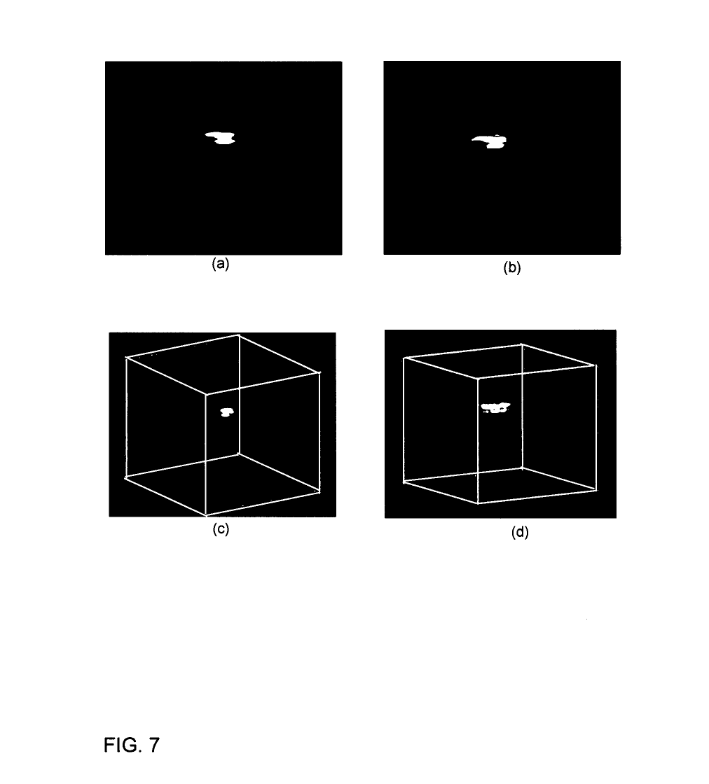

Photoacoustic imaging data was acquired by recording an element's output after the laser excitation. The individual element acquisitions were bandpass filtered and then used for image reconstruction. The data was averaged 4 times to improve the signal-to-noise ratio. An example of photoacoustic data acquisition is shown in FIG. 6. The signal from the ink-filled tube can be clearly seen. The signals seen in the first five microseconds are due to electronic noise of the laser and laser light incident on the transducer array. Photoacoustic images of the phantom are shown in FIG. 7. The photoacoustic images were constructed using a standard delay and sum image reconstruction algorithm. FIGS. 7 (a) and (b) are XZ and YZ slices, respectively, taken from a 3D photoacoustic image with 15 dB dynamic range. FIG. 7(c) shows a volume rendered photoacoustic image of the phantom. FIG. 7(d) illustrates the increased clarity resulting from array tiling. The ink-filled tube can be clearly seen to curve upward in this volume rendered image.

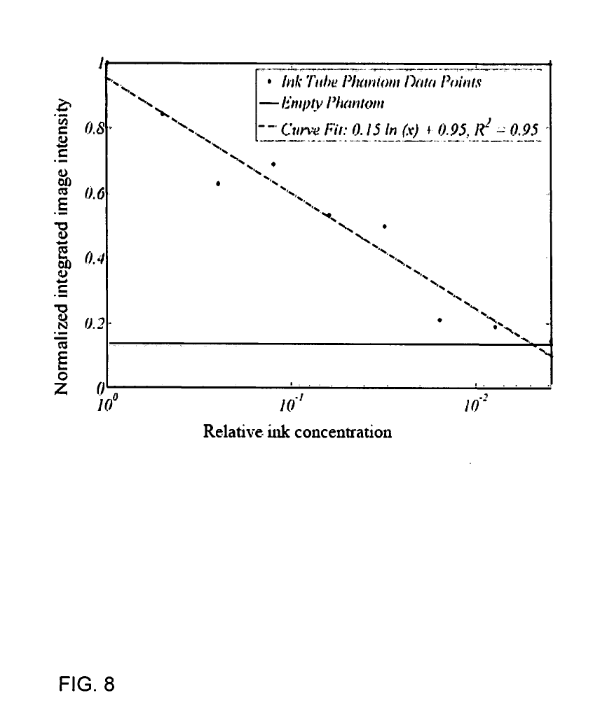

To investigate the sensitivity of the system, an experimental setup similar to that described above was used, but the phantom was made of one 1.14-mm inner diameter (1.57-mm outer diameter) polyethylene tube passing through a 4 cm×4 cm×4 cm block of tissue mimicking material (ATS Laboratories, Bridgeport, Conn.). The phantom was positioned such that the tube was 2 cm above the CMUT array and filled with India-ink to provide optical contrast for the photoacoustic imaging. The concentration of the India ink was varied in powers of ½ and images were taken. A simple integration of the pixel values in a volume surrounding the ink-tube was performed on each image. These values were then normalized. Results from this experiment are summarized in the graph shown in FIG. 8.

As one of ordinary skill in the art will appreciate, various changes, substitutions, and alterations could be made or otherwise implemented without departing from the principles of the present invention. Accordingly, the scope of the invention should be determined by the following claims and their legal equivalents.

What is claimed is:

1. An apparatus for functional ultrasound imaging of an object, comprising:
a) a source of non-ultrasonic excitation energy, wherein said source induces generation of ultrasonic waves within said object; and
b) a single capacitive micromachined ultrasonic transducer (CMUT) or an array of CMUTs, wherein said single CMUT or said array of CMUTs is situated to detect said generated ultrasonic waves.

2. The apparatus as set forth in claim 1, wherein said source is an optical fiber, a vertical cavity surface emitting laser, a microfabricated electron beam source, or a nanokylstron.

3. The apparatus as set forth in claim 1, wherein said array of CMUTs is configured in 1 dimension or in 2 dimensions.

4. The apparatus as set forth in claim 1, wherein said array of CMUTs is configured as an annular ring array, an annular array, a linear array, or a rectangular array.

5. The apparatus as set forth in claim 1, wherein said array of CMUTs is formed on a curved surface or around said object.

6. The apparatus as set forth in claim 1, wherein said array of CMUTs has elements along each dimension that measure about one-half a wavelength of said generated ultrasonic waves.

7. The apparatus as set forth in claim 1, wherein said apparatus further comprises integrated circuitry.

8. The apparatus as set forth in claim 1, wherein said source and said CMUT array are integrated on one substrate.

9. A method of functionally imaging an object, comprising:
a) exposing said object to a source of non-ultrasonic energy, wherein said source induces generation of ultrasonic waves within said object; and
b) detecting said generated ultrasonic waves with a single capacitive micromachined ultrasonic transducer (CMUT) or an array of CMUTs.

10. The method as set forth in claim 9, wherein said object further comprises at least one contrast agent.

11. The method as set forth in claim 9, further comprising observing intensity of said generated ultrasonic waves as a function of excitation frequency of said source.

12. The method as set forth in claim 9, further comprising ablating tissue with said source.

13. The method as set forth in claim 9, further comprising monitoring said ablating.

14. The method as set forth in claim 9, further comprising coding an excitation scheme of said exposing and decoding a signal generated by said detected ultrasonic waves.

15. A method of functionally and mechanically imaging an object, comprising:
a) exposing said object to a source of non-ultrasonic energy, wherein said source induces generation of ultrasonic waves within said object;
b) detecting said generated ultrasonic waves with an array of CMUTs, wherein said array comprises two or more CMUTs;
c) transmitting ultrasonic waves through said object using one or more of said CMUTs of said array;
d) detecting said transmitted ultrasonic waves with one or more of said CMUTs of said array;
e) processing signals detected by said array of CMUTs to form an image from said detecting of said generated ultrasonic waves and to form an image from said detecting of said transmitted ultrasonic waves; and
f) displaying said images either separately or as overlapping images.

16. The method as set forth in claim 15, wherein said object further comprises at least one contrast agent.

17. The method as set forth in claim 15, further comprising observing intensity of said generated ultrasonic waves as a function of excitation frequency of said source.

18. The method as set forth in claim 15, further comprising ablating tissue with said source.

19. The method as set forth in claim 15, further comprising monitoring said ablating.

20. The method as set forth in claim 15, further comprising coding an excitation scheme of said exposing and decoding a signal generated by said detected ultrasonic waves.
